(19) United States
(12) Patent Application Publication
(10) Pub. No.: US 2013/0052169 A1
(43) Pub. Date: Feb. 28, 2013

(54) STEM CELL BANK FOR PERSONALIZED MEDICINE

(71) Applicant: Ehud Marom, Kfar Saba (IL)

(72) Inventor: Ehud Marom, Kfar Saba (IL)

(73) Assignee: STEM CELL MEDICINE LTD., Ness Ziona (IL)

(21) Appl. No.: 13/696,383

(22) PCT Filed: May 5, 2011
(86) PCT No.: PCT/IL11/00362

§ 371 Date: Nov. 6, 2012

Related U.S. Application Data
(60) Provisional application No. 61/429,773, filed on Jan. 5, 2011.
(60) Provisional application No. 61/331,842, filed on May 6, 2010.

(51) Int. Cl. A61K 35/12; A61K 35/30; A61K 35/50; A61K 35/14; A61K 35/48; A61K 35/34; C12N 5/071; C12N 5/0789; C12N 5/0797; C12N 5/0775; C12N 5/074; A61P 7/00; A61P 25/00; A61P 9/00; A61K 35/54

(52) U.S. Cl. 424/93.7; 435/325; 435/374

(57) ABSTRACT

A stem cell bank is disclosed which stores stem cells collected from individuals throughout their entire life. The stem cell bank stores stem cells of various types, which are obtained from a plurality of sources from a single individual. Also provided are methods of personalized medicine that utilizes cells stored in a bank and compositions of stem cells for the treatment of various types of diseases.

### FIELD OF THE INVENTION

The present invention relates to a stem cell bank for accumulation of different types of stem cells from an individual over the course of the lifetime of said individual. The stem cell bank enables treatment of a disease or disorder with combinations of stem cells having cells of different types. Combinations of stem cells in different ratios may be used for specific diseases and tissues regeneration.

### BACKGROUND OF THE INVENTION

Stem cells are cells found in all multi-cellular organisms. They are characterized by the ability to renew themselves through mitotic cell division and differentiate into a diverse range of specialized cell types. Due to these unique properties, stem cells are considered to be of special interest in the field of cell therapy, particularly when replacements for lost or damaged cells are required.

In mammals, two major categories of stem cells are identified: embryonic stem cells and adult stem cells.

Embryonic stem cells (ESC) are derived from blastocysts which arise in a very early stage of embryonic development. ESCs can develop into each of the more than 200 cell types of the adult body when given sufficient and necessary stimulation for a specific cell type. ESCs can be grown in culture to large numbers but are difficult to control in their development and are accompanied by ethical problems.

Adult stem cells (ASC) are found in various tissues of the adult body. Each tissue and organ in the body originates from a small number of ASCs which are committed to differentiate into the various cell types that compose the tissue. The primary roles of adult stem cells in a living organism are to maintain and repair the tissue in which they are found. Since stem cells are continued to be produced throughout the entire life of an individual (even though a significant decrease in production occurs with age) it is possible to obtain ASCs from a newborn, a child, or an adult. Within the category of adult stem cells, many types of cells are known and they are generally referred to by their tissue origin. For example, hematopoietic stem cells (HSC) which form the basis for most, if not all, blood cells, and reconstitute the immune system. HCSs are located in the bone marrow, the circulation and other organs. Additional example is mesenchymal stem cells (MSC), that can differentiate into bone, cartilage, fat, tendon, muscle, connective tissue and marrow stroma. Under suitable conditions they can give rise to additional tissues such as blood vessels, liver and nerve cells, as well as insulin secreting Langerhans cells. Other examples include adipose-derived stem cells and endothelial stem cells. In fact, every tissue and organ in the body is likely to contain stem cells that participate in intrinsic regeneration and repair during growth, trauma and disease.

Adult stem cell treatments have been successfully used for many years to treat leukemia and related bone/blood cancers, anemia and immune system dysfunctions through bone marrow transplantations. Adult stem cells are also used in veterinary medicine to treat tendon and ligament injuries in horses. Many additional disease indications, such as ischemic heart diseases, neural injuries, neurodegenerative diseases and diabetes, are currently under investigation at their pre-clinical research stage.

Another category of stem cells is induced pluripotent stem cells (IPSC), which are pluripotent stem cells artificially derived from a non-pluripotent cell. Typically, IPSCs are derived from adult somatic cells, using genetic or epigenetic manipulations. Such cells are believed to be identical to natural pluripotent stem cells, such as embryonic stem cells in many respects, for example in terms of the expression of certain stem cell genes and proteins, chromatin methylation patterns, doubling time, embryoid body formation, teratoma formation, viable chimera formation, and potency and differentiability. However, the full extent of their relation to natural pluripotent stem cells is still to be assessed. Induced pluripotent stem cells have generated interest for regenerative medicine, as they allow the production of patient-specific progenitors in vitro with potential value for cell therapy.

Stem cells transplants can be either autologous or allogeneic. An autologous stem cell transplant is one in which the patient receives stem cells from his own body. One advantage of this stem cell treatment procedure is that the body recognizes the cells and therefore does not reject or attack them, an occurrence known as Graft-Versus-Host Disease (GVHD). An allogeneic stem cell transplant is a procedure in which a patient receives stem cells from a donor. The donor used in an allogeneic stem cell transplant can be the patient's identical twin, sibling, family member or an unrelated donor. Allogeneic transplants are preferred in certain cases, such as in the treatment of leukemia.

Even though adult stem cells hold a large potential as therapeutic agents, some limitations to their use still exist. One of the basic limitations is their scarce availability in adults. Despite advances in their procurement, obtaining sufficient quantities and populations of human stem cells capable of differentiating into the many different desired cell types still remains a challenge. In addition, several clinical trials of stem cells treatments have shown statistically significant but moderate results, thus raising a demand for optimization of the processes.

The advances in the procurement and use of stem cells have led to a need for storage repositories, also termed stem cell banks, for storing such cells. Known adult stem cell banks can generally be classified into two categories, private banks and public banks. Private banks collect and store autologous adult stem cells and provide a unit of the donated stem cells back to the donor if needed. Public banks provide typed, anonymous transplant units to the general public based on genetic matching between a donor and a recipient in need. Current banks mainly specialize in collecting cells from the peripheral blood of adults and from the umbilical cord blood of healthy new born infants. At present, commercial stem cell banks offer only limited types of stem cells and limited amount of donations.

U.S. Pat. No. 5,993,387 discloses a computer-based mixed-use cord stem cell registry system, method and device, for developing and maintaining a mixed-used bank of placental and umbilical cord stem cells and for the resultant bank. The cord stem cells, or a fraction thereof, are stored in a bank for the potential use of the donor and potentially the actual family of the donor child or from an unrelated person for whom the cord stem cells are a match.

U.S Patent Application Publication No. 2004/0091936 discloses methods for producing stem cell banks, preferably human, which optionally may be transgenic, e.g., comprised of homozygous MHC allele cell lines. These cells are produced preferably from parthenogenic, IVF, or same-species or cross-species nuclear transfer embryos or by de-differentiation of somatic cells by cytoplasm transfer. Methods for using these stem cell banks for producing stem and differentiated cells for therapy, especially acute therapies, and for screening for drugs for disease treatment are also disclosed.

U.S Patent Application Publication No. 2005/0276792 discloses systems and methods for enabling a stem cell bank to provide individual transplant units of stem cells to patients. Advantageously, such stem cell transplant units can be from a single donor.

WO 2007/024441 discloses compositions and method for the preparation and use of mixtures of adult stem/progenitor cell populations recovered and enriched from specific tissues with very limited attempts for their purification. Such mixtures of cell populations have improved therapeutic effectiveness in the treatment of certain diseases and tissue regeneration treatments over their more purified counterpart cell populations. Such mixtures of cell populations can be cryopreserved for future clinical use.

U.S Patent Application Publication No. 2008/0102521 discloses methods for producing a stem cell bank and providing stem cell samples for purchase or use. Also provided are embodiments relating to a stem cell bank and stem cell banking system.

WO 2009/152485 discloses methods relating to conducting a stem cell technology business such as a regenerative medicine business based on induced pluripotent stem cells (iPSCs) and cells differentiated from iPSCs. The disclosure also provides a database of iPSC-derived cells and methods of using the database for tracking customers and samples, as well as methods for marketing and running the business.

WO 2010/033969 discloses methods of inducing pluripotency in cells, methods of identifying pluripotent cells and methods of culturing pluripotent cells. The present invention further encompasses methods of making pluripotent, autologous, patient-specific, cell banks derived from amniotic fluid cells.

WO 2010/148334 discloses methods and compositions for the generation and use of genetically corrected induced pluripotent stem cells. It is disclosed that using certain methods and compositions, cord blood (CB) stem cells can be reprogrammed to pluripotency by retroviral transduction with OCT4, SOX2, KLF4, and c-MYC, in a process that is extremely efficient and fast. It is also disclosed that the described methods and compositions may set the basis for the creation of a comprehensive bank of HLA-matched CBiPS cells for off-the-shelf applications.

There is an unmet need to increase the availability of stem cells of different types, and the efficiency of stem cells treatments. It would be highly beneficial to have a system that will guarantee a sufficient amount of available stem cells from different sources for therapeutic and research applications, which may offer potential treatment for many types of diseases, as well as an optimized treatment when a person is in need of cell therapy.

### SUMMARY OF THE INVENTION

The present invention provides a stem cell bank which stores stem cells collected from individuals throughout their entire life. The stem cell bank of the present invention stores stem cells of various types, which are obtained from a plurality of sources from a single individual. Thus, the stem cell bank of the present invention provides a large pool of available stem cells, that may be utilized in a variety of therapeutic, as well as research, applications. The stored stem cells may serve, for example, as a source of cells for use in the future when health reasons require stem cells technologies to treat certain cell populations of an individual's body. The stored stem cells may serve as a source of cells for autologous use, for example, for curing future diseases of a donor. The stored stem cells may also serve as a source of cells for clinical use by other individuals upon authorization from the donor. In some embodiments, the bank provides storage and management of an individual and family “insurance” through periodical self donations of stem cells from various sources. The present invention further provides methods of personalized medicine that utilizes cells stored in a bank of the present invention.

The present invention further provides compositions of stem cells for the treatment of various types of diseases. The invention discloses a combination of embryonic and adult stem cells as a “booster dose” when applying stem cell treatment. The invention further discloses a combination of adult stem cells of various types, mixed in different ratios and doses and formulations, in order to achieve maximum therapeutic effect. For example, adult stem cells may be derived from hematopoietic origin, placenta, oral mucosa, mesenchymal sources and fat-derived stem cells, as are known in the art. The combinations of stem cells from several sources are now disclosed to have improved therapeutic effectiveness compared to existing stem cells compositions.

According to one aspect, the present invention provides a stem cell bank comprising stem cells from a plurality of individuals, wherein the stem cells originate from a plurality of donations collected periodically from each individual throughout the individual's life.

In some embodiments, a stem cell bank is provided, wherein a plurality of donations are collected periodically from an individual throughout the individual's life, sorted and stored for future use.

In some embodiments, the plurality of donations collected from said each individual are obtained from different sources.

In some embodiments, the plurality of donations collected from said each individual comprises stem cells of different types.

As used herein, a “donation” refers to a sample or samples of cells collected from a subject at a certain time point. One donation may include samples of cells collected from a single source or samples of cells collected from multiple sources. Each sample may include collection of cells of the same type, or collection of cells of more than one type. The donation may include stem cells, as well as differentiated somatic cells. The differentiated somatic cells may be used to generate induced pluripotent stem cells (IPSCs), as described below.

As used herein, “donations collected periodically” or “periodical donations” refer to donations collected from an individual at predetermined time intervals, for example, every 5 years, every 10 years, every 15 years and the like. Alternatively, “donations collected periodically” or “periodical donations” refer to collecting donations from an individual at certain time points, for example, at particular ages.

In some embodiments, the plurality of donations comprises a first donation at birth and subsequent donations as the individual grows and matures. In some typical embodiments, donations, or subsequent donations, are collected between the ages 20 to 50.

In some embodiments, a first donation is collected at birth, for example, from umbilical cord blood and/or placental blood. In some embodiments, a first donation is collected before birth, for example, from amniotic fluids.

In general, every tissue and organ that contains stem cells may be a source of stem cells for donation. In some embodiments, the source of stem cells comprises at least one source selected from the group consisting of umbilical cord blood, cord matrix, placental blood, bone marrow, fat, peripheral blood, blood buffy coat, amniotic fluid, skin, kidney, liver, muscle, neural tissue, tooth pulp, mucosa (including but not limited to oral, olfactory and gastric), foreskin, cardiac tissue, bone, cartilage, hair roots and mammary glands. Each possibility represents a separate embodiment of the invention.

In some embodiment, the donations comprise differentiated somatic cells. Such cells may be used to generate induced pluripotent stem cells (IPSCs), as described below.

In some embodiments, the differentiation potential of the stem cells stored in the bank is selected from the group consisting of pluripotent, multipotent, oligopotent and unipotent. Each possibility represents a separate embodiment of the invention.

In some embodiments, the stem cells stored in the bank comprise at least one type selected from the group consisting of hematopoietic cells, lineage-committed hematopoietic cells, mesenchymal stem cells, stromal cells, fibroblasts, endothelial progenitor cells, neural stem cells, adipose-derived stem cells, stem cells derived from mucosa, placenta-derived stem cells, amniotic stem cells, cord blood derived stem cells, cord matrix derived stem cells, stem cells derived from foreskin, cardiac stem cells and mammary stem cells. Each possibility represents a separate embodiment of the invention.

In some embodiments, information about each donation is recorded. In some specific embodiments, the recorded information comprises at least some data selected from the group consisting of the type of cells, their tissue of origin, the date of their collection and the identity of the donor. In other specific embodiments, the recorded information comprises results obtained from various characterization assays. Examples include HLA typing, determining the presence of specific markers, determining specific SNP alleles and/or performing a nucleated cell count on the stem cell unit.

In some embodiments, the collected cells are sorted according to at least one criterion. In some specific embodiments, they are sorted according to their type, their tissue of origin, the date of their collection and the donor identity.

In some embodiments, the collected stem cells are stored under appropriate conditions to keep the stem cells viable and functional. In some specific embodiments, the stem cells are stored under cryopreservation conditions.

In some embodiments, stem cells stored in the bank are for autologous use. In some embodiments, the stored stem cells are used for autologous transplantations.

In other embodiments, stem cells stored in the bank are for allogeneic use. In some embodiments, the stored stem cells are used for allogeneic transplantations. In other embodiments, the stored stem cells are used for the establishment of cell lines having, for example, good viability and other desirable characteristics for research and pharmaceutical applications.

In some embodiments, the stem cells stored in the bank are arranged in stem cell units. According to these embodiments, each donation to the bank (each deposit of stem cells) is divided into a plurality of stem cell units. In some typical embodiments, a stem cell unit comprises a population of stem cells of the same type that were collected from a single donor in a single donation, or a population of induced pluripotent stem cells of the same type generated from cells collected from a single donor in a single donation. In some exemplary embodiments, a stem cell unit includes stem cells expressing a specific marker or markers. In some embodiments, a stem cell unit is further defined by the number of nucleated cells present in the sample. Upon request, one or more stem cell units may be allocated to a subject in need thereof. In some embodiment, a fraction of a stem cell unit is allocated to a recipient in need. In some typical embodiments, the number of stem cell units to be allocated depends on the number of nucleated cells in each unit and the medical condition to be treated.

In some embodiments, the amount of stem cells, or the number of stem cell units, available for allocation to an individual depends on the amount of donations made by that individual.

In some embodiments, the stem cells can be subjected to further processing after their collection. In some specific embodiments, the collected stem cells can be cultured, expanded and/or proliferated. In additional specific embodiments, the collected stem cells are processed in order to achieve therapeutic levels.

In some embodiments, an optimal combination of stem cells types can be selected from the reservoir of cells, in order to treat a certain pathological condition.

According to another aspect, the present invention provides a method of stem cell banking, the method comprising periodically collecting a plurality of donations from an individual throughout the individual's life.

In some embodiments, the method comprises collecting stem cells from more than one source. In some embodiments, the method comprises collecting stem cells of more than one type. In some embodiments, the method comprises collecting somatic cells.

In some embodiments, the method further comprises dividing each donation to stem cell units.

In some embodiments, stem cells stored in the bank serve as a basis for personalized medicine. In some specific embodiments, the cells form the basis for a personalized medicine which is based on the ability to cure and regenerate parts of the body that no longer function and/or were damaged by new cells that replace the damaged ones.

Thus, according to another aspect, the present invention provides a method of personalized medicine, the method comprising providing stem cells stored in a stem cell bank of the present invention, wherein said stem cells originate from a single individual; and administering said stem cells to said individual.

In some embodiments, the stem cells undergo further processing before they are administered to the individual. For example, the cells may be subjected to differentiation procedures.

The provided stem cells may be the same or different. In some embodiments, the provided stem cells are of the same type. In other embodiments, a combination of stem cells of different types is used.

In some embodiments, a composition for use in personalized medicine is provided, the composition comprising stem cells reconstituted from a bank of the present invention.

The variety of stem cells types stored in the bank, together with periodical donations, confer a wide option for protection against many pathological conditions that require organ repair, and might be encountered by an individual in the course of the individual's life.

According to another aspect, the present invention provides a composition comprising stem cells mixtures for use in stem cell therapy.

According to yet another aspect, the present invention provides the use of a composition comprising stem cells mixtures in stem cell therapy.

In some embodiments, the composition comprises mixtures of adult stem cells of more than one type. In some embodiments, the composition of stem cells comprises embryonic and adult stem cells. In some embodiments, the composition comprises adult stem cells of more than one type, mixed in different ratios. In additional embodiments, the composition of stem cells comprises adult stem cells of various types, wherein each type is present in a different dose.

In various specific embodiments, the adult stem cells in the composition can be of various types. Any type of adult stem cells known in the art can be used in the compositions of the present invention. In some embodiments, the types of adult stem cells in the compositions comprise at least one type selected from the group consisting of hematopoietic cells, lineage-committed hematopoietic cells, mesenchymal stem cells, stromal cells, fibroblasts, endothelial progenitor cells, neural stem cells, adipose-derived stem cells, stem cells derived from mucosa, placenta-derived stem cells, amniotic stem cells, cord blood derived stem cells, cord matrix derived stem cells, stem cells derived from foreskin, cardiac stem cells and mammary stem cells. Each possibility represents a separate embodiment of the invention. Any combination comprising at least two types of stem cells may be useful for the compositions of the present invention.

In some embodiments, the composition of stem cells comprises induced pluripotent stem cells. Any type of IPSC can be used in the compositions of the present invention.

In some embodiments, the composition of stem cells comprises IPSCs of more than one type, mixed in different ratios. In some additional embodiments, the composition of stem cells comprises IPSCs of various types wherein each type is present in a different dose.

In some specific embodiments, the types of stem cells to be mixed and the ratio between them are determined and optimized according to the pathological condition of an individual in need. It is contemplated that by optimizing the ratio between the different types of stem cell in a composition, an improved therapeutic effectiveness could be achieved.

The compositions of the present invention may be provided in various dosage forms. The various dosage forms include, but are not limited to, liquid dosage forms, for example, for injection.

In some embodiments, the differentiation potential of the stem cells present in the compositions of the present invention is selected from the group consisting of pluripotent, multipotent, oligopotent and unipotent.

In some embodiments, the adult stem cells in the compositions are of an autologous origin. In other embodiments, the adult stem cells in the compositions are of an allogeneic origin.

In some embodiments, the IPSCs in the compositions are of an autologous origin. In other embodiments, the IPSCs in the compositions are of an allogeneic origin.

In some embodiments, the stem cells were obtained from periodical donations collected during the course of life of the individual in need.

These and further aspects and features of the present invention will become apparent from the detailed description and claims which follow.

### DETAILED DESCRIPTION OF THE INVENTION

The present invention provides a stem cell bank which stores stem cells obtained from individuals over the course of the individuals' life. The stored stem cells may serve as a source of cells for use in the future when health reasons require stem-cells technologies to fix certain cell populations of the individual's body, as well as for clinical use by other individuals. The stored stem cells may also be used for research applications. The present invention further provides compositions of stem cells for the treatment of various types of diseases.

### Definitions

“Stem cell therapy”—as used herein, refers to all of the uses known or envisioned in the art for stem cells. These uses include diagnostic, prophylactic and therapeutic techniques.

“Bank”, “Stem cell bank”—used interchangeably, refer to a repository of stem cells, wherein upon request, demand and/or need the stored stem cells can be recovered from storage and allocated to a certain individual for a certain clinical purpose. Alternatively or additionally, the stored stem cells can be used for research applications.

“Somatic cell”—any cell other than a germ cell or a germ cell precursor.

“Totipotent stem cell”—a stem cell capable of differentiating into any cell type of an organism's body, including germ line cells. Examples of totipotent cells include an embryonic stem cell, an embryonic germ cell, an inner cell mass (ICM)-derived cell, or a cultured cell from the epiblast of a late-stage blastocyst. A totipotent stem cell is able to develop into a complete organism.

“Pluripotent stem cell”—a stem cell capable of generating the three embryonic germ layers (endoderm, mesoderm and ectoderm) and the cell lineages, tissues and organs originating from these layers.

“Multipotent stem cell”—a stem cell capable of forming multiple cell lineages generally derived from one embryonic germ layer.

“Oligopotent stem cell”—a stem cell that can differentiate into only a few cells, such as lymphoid or myeloid stem cells.

“Unipotent stem cell”—a stem cell that can produce only one cell type, its own, but have the property of self-renewal which distinguishes it from non-stem cells (e.g. muscle stem cells).

“Induced pluripotent stem cell”—a pluripotent stem cell artificially derived from a non-pluripotent cell. A non-pluripotent cell may be a fully differentiated cell or a cell whose potency to self-renew and differentiate is lower than that of a pluripotent stem cell. Typically, induced pluripotent stem cells are derived from adult somatic cells.

### Stem Cell Bank

The stem cell bank of the present invention can be considered as a first component of a system aimed at providing a sufficient amount of available stem cells for potential treatment of many types of diseases.

In some embodiments, the bank of the present invention offers a “self-insurance” program for an individual and/or a family by periodical self-donations of adult stem cells from various sources.

According to one aspect, the present invention provides a stem cell bank, wherein a plurality of donations are collected periodically from an individual throughout the individual's life.

In some embodiments, a donation, or deposit, comprises cells obtained from one source. In other embodiments, a donation comprises cells obtained from more than one source.

According to some embodiments, a plurality of donations is collected throughout an individual's life. In some embodiments, the plurality of donations comprises a first donation at birth and subsequent donations as the individual grows and matures. In general, it is possible to obtain adult stem cells from a newborn, a child, or an adult. Optimally, the subsequent donations are collected from individuals of ages 20 to 50. It is to be understood that donations may be collected before and/or after these ages.

In some embodiments, differentiated somatic cells are collected from the individual. Such cells may be induced to generate pluripotent stem cells.

In some embodiments, the stored stem cells are for autologous use. According to these embodiments, periodical self donations are collected throughout the individual's life. In other embodiments, the stored stem cells are for allogeneic use.

Allogeneic use may include allogeneic transplantations, as well as research applications. Samples are typically taken from each donation (each deposit of stem cells to the bank) to be screened for viability and lack of contamination prior to storage. These samples may be used to establish a reservoir or stockpile of stem cells for allogeneic uses. It will be appreciated that normally, an authorization from the donor is required in order to allocate stem cells obtained from that donor for allogeneic uses. The reservoir of stem cells for allogeneic uses may be used to establish cell lines from cells having good viability and other desirable characteristics. In some embodiments, selection processes are employed in order to isolate optimized cells for the establishment of cell lines.

The number of donations and the sources from which samples are collected each time may be determined according to a standard program. In some embodiments, a specific, personal program is determined for each individual. In some exemplary embodiments, a personal program is defined for individuals who are at risk for developing a certain disease (for example, based on family medical background).

In some embodiments, donations are collected at periodic intervals. A periodic interval for the collection of donations may range from 1-5 years, 5-10 years, 10-15 years. Each possibility represents a separate embodiment of the invention.

In some embodiments, donations are collected at predetermined time points. A first donation may be collected at birth, during childhood or at adulthood. Each possibility represents a separate embodiment of the invention.

In some embodiments, the number of donations collected from one individual ranges from 2-5, 2-10, more than 5, more than 10. Each possibility represents a separate embodiment of the invention.

According to another aspect, the present invention provides a method of stem cell banking, the method comprising providing a plurality of periodical donations from an individual throughout the individual's life.

In some embodiments, a method for maintaining a stem cell bank is provided, the method comprising periodically collecting a plurality of donations from an individual throughout the individual's life.

In some embodiments, the method comprises providing stem cells from more than one source. In some embodiments, the method comprises providing stem cells of more than one type. In some embodiments, the method comprises providing somatic cells.

In some embodiments, the method further comprises dividing each donation to stem cell units.

### Procedures for the Collection of Stem Cells

For the purpose of the present invention, every tissue and organ that contains stem cells can be a potential source of stem cells to be extracted and stored in the bank. These include sources which are currently known in the art as well as new sources that may be discovered in the future.

Non-limiting examples of sources include umbilical cord blood, placental blood, bone marrow, fat, peripheral blood, cord matrix, blood buffy coat, amniotic fluid, ascitic fluid, skin, kidney, liver, muscle, neural tissue, tooth pulp, oral mucosa, olfactory mucosa, gastric mucosa, foreskin, cardiac tissue, bone, cartilage hair roots and mammary glands.

The present invention encompasses any known method of acquiring stem cells from donors.

Stem cells can be recovered from a sample by extraction. Many extraction methods are known in the art and can be used for extracting stem cells to be stored in the bank of the present invention. Suitable cell extraction methods include, but are not limited to: plasmapheresis, centrifugation at defined time and g-force or density gradient centrifugation, centrifugation following the addition of some fluids such as physiological solutions or certain soluble polymers, cellular adherence to plastic, and adherence to reagents used to coat growth surfaces including reagents such as fibronectin, and collagen. In addition, mechanical cell sorting methods can be used and enzymatic methods can be used, as are known.

Separation of the stem, cells may be performed according to various physical properties, such as fluorescent properties or other optical properties, magnetic properties, density, electrical properties, etc. Cell types can be isolated by a variety of means including fluorescence activated cell sorting (FACS), protein-conjugated magnetic bead separation, morphologic criteria, specific gene expression patterns (using RT-PCR), or specific antibody staining.

The use of separation techniques includes, but is not limited to, techniques based on differences in physical (density gradient centrifugation and counter-flow centrifugal elutriation), cell surface (lectin and antibody affinity), and vital staining properties (mitochondria-binding dye rho123 and DNA-binding dye Hoechst 33342).

Cells may be selected based on light-scatter properties as well as their expression of various cell surface antigens. The purified stem cells have low side scatter and low to medium forward scatter profiles by FACS analysis.

Various techniques can be employed to separate the cells by initially removing cells of dedicated lineage. Monoclonal antibodies are particularly useful. The antibodies can be attached to a solid support to allow for crude separation. The separation techniques employed should maximize the retention of viability of the fraction to be collected.

The separation techniques employed should maximize the retention of viability of the fraction to be collected. Various techniques of different efficacy may be employed to obtain “relatively crude” separations. Such separations are where up to 30%, usually not more than about 5%, preferably not more than about 1%, of the total cells present are undesired cells that remain with the cell population to be retained. The particular technique employed will depend upon efficiency of separation, associated cytotoxicity, ease and speed of performance, and necessity for sophisticated equipment and/or technical skill.

Procedures for separation may include magnetic separation, using antibody-coated magnetic beads, affinity chromatography, cytotoxic agents joined to a monoclonal antibody or used in conjunction with a monoclonal antibody, e.g., complement and cytotoxins, and “panning” with antibody attached to a solid matrix, e.g., plate, or other convenient technique.

Techniques providing accurate separation include fluorescence activated cell sorters, which can have varying degrees of sophistication, e.g., a plurality of color channels, low angle and obtuse light scattering detecting channels, impedance channels, etc.

Other techniques for positive selection may be employed, which permit accurate separation, such as affinity columns, and the like.

Antibodies used for separation may be conjugated with markers, such as magnetic beads, which allow for direct separation, biotin, which can be removed with avidin or streptavidin bound to a support, fluorochromes, which can be used with a fluorescence activated cell sorter, or the like, to allow for ease of separation of the particular cell type. Any technique may be employed which is not unduly detrimental to the viability of the remaining cells.

Exemplary procedures for the isolation and characterization of stem cells from various sources can be found, for example, in Lanza (ed.) Handbook of stem cells, Volume 2, Gulf Professional Publishing, 2004; Fierabracci (2010) Recent Pat Drug Deliv Formul, 4(2):105-13; Brignier et al. (2010) J Allergy Clin Immunol, 125(2 Suppl 2):S336-44.

In some embodiments, a population of sorted stem cells of the same type originating from a single donation from a single donor defines a stem cells unit. In some embodiments, a stem cell unit is further defined by the number of nucleated cells present in the unit.

The number of nucleated cells present in the unit may range from 25*10⁷-50*10⁷, from 50*10⁷-75*10⁷, from 75*10⁷-200*10⁷, from 150*10⁶-10,000*10⁶, from 300*10⁶-5,000*10⁶, from 500*10⁶-3,000*10⁶.

Non-limiting examples of cell types which can be collected and stored in the bank include hematopoietic cells, lineage-committed hematopoietic cells, mesenchymal stem cells, stromal cells, fibroblasts, endothelial progenitor cells, neural stem cells, stem cells derived from mucosa, placenta-derived stem cells, amniotic stem cells, cord blood derived stem cells, fat source stem cells, stem cell derived from foreskin, cardiac muscle stem cells and mammary stem cells.

In some embodiment, differentiated somatic cells are collected from the individual.

In some embodiments, the obtained cells are used to generate induced pluripotent stem cells. It is to be understood that the induction to pluripotency may be performed for differentiated somatic cells, as well as for stem cells with a low differentiation potential.

Various methods for the induction of pluripotency are known in the art, which can be applied for various types of cells. Some of the known methods include genetic manipulations, for example transfection of certain stem cell-associated genes using retroviruses, and epigenetic manipulations, for example direct delivery of reprogramming proteins into the cells.

As a non-limiting example, induced pluripotent stem cells (IPSCs) may be produced from dermal fibroblasts, as described, for example, in Takahashi et al. (2007) “Induction of pluripotent stem cells from adult human fibroblasts by defined factors”, Cell, Vol. 131, pp. 1-12.

As an additional non-limiting example, IPSCs may be produced from T cells and/or hematopoietic progenitor cells, as described, for example, in International Patent Application Publication No. WO 2010/141801.

A non-limiting example for the induction of IPSCs by the delivery of certain reprogramming factor proteins into cells can be found in International Patent Application Publication No. WO 2010/115052.

Thus, in some embodiments, induced-pluripotent stem cells are stored in the bank.

In some embodiments, the stem cell bank of the present invention stores lines of induced pluripotent stem cells derived from various cell types.

The large variety of stem cells and somatic cells stored in the bank, that are obtained from many donors and from various tissue sources, creates a large pool of cells, from which the best cells can be isolated. In some embodiments, selection processes are employed in order to isolate optimized cells for the establishment of cell lines. Selection processes may also be employed for the isolation of optimized cells for use as a source for the generation of IPSCs. For example, selection procedures may be employed that promote the isolation of cells with increased stability.

Procedures for selection of cell lines in general and/or optimized cells for further manipulation are within the knowledge of one of skill in the art. The suitable selection process may be chosen according to the cells type, and may be performed by methods well known in the art.

In some embodiments, the optimized cell lines are used for research applications, including but not limited to, drug development and testing.

In some embodiments, the differentiation potential of the stem cells stored in the bank can be selected from pluripotent, multipotent, oligopotent and unipotent.

In some embodiments, the differentiation potential of the stem cells stored in the bank is other than totipotent.

In some embodiments, according to an individual's request, gametes are collected from that individual and stored in the bank.

The obtained stem cells can be characterized by various methods. In some embodiments, characterization includes the results of various assays. In some specific embodiments, the characterization comprises a test for the presence of specific markers, according to the desired population of cells. In additional specific embodiments, the characterization comprises the determination of the human leukocyte antigen (HLA) type. In yet additional specific embodiments, the characterization comprises the determination of specific SNP alleles. In yet additional specific embodiments, the characterization comprises performing a nucleated cell count on the stem cell unit.

The information obtained may include genotype or phenotype information. Phenotype information may include any observable or measurable parameter, either at a macroscopic or system level or microscopic or even cellular or molecular level. Genotype information may refer to a specific genetic composition of a specific individual organism, for example, whether an individual organism has one or more specific genetic variants up to all the variations in that individual's genome, for example, whether the individual is a carrier of genetic variations that influence disease or the HLA type of that individual.

Any method known in the art for the characterization of stem cells may be appropriate for the purposes of the present invention.

The obtained stem cells can be subjected to further processing. The collected cells can be processed with different technologies that exist today and new technologies that may be developed in the future.

In some embodiments, after stem cells have been obtained from certain tissues of the donor, they are cultured using stem cell expansion techniques.

Stem cell expansion techniques are disclosed, for example, in U.S. Pat. No. 6,326,198, U.S. Pat. No. 6,338,942 and U.S. Pat. No. 6,335,195.

Thus, in some embodiments, stem cells obtained from the donor are cultured in order to expand the population of stem cells.

Additional processing methods known in the art include, for example, those disclosed in U.S. Pat. No. 6,059,968 and U.S. Pat. No. 5,879,318. In some embodiments, processing prepares the stem cell products for storage or for further use.

An optional procedure is to expand the stem cells in vitro. However, care should be taken to ensure that growth in vitro does not result in the production of differentiated progeny cells at the expense of multipotent stem cells which are therapeutically necessary for reconstitution. Various protocols have been described for the growth in vitro of cord blood or bone marrow cells, and it is envisioned that such procedures, or modifications thereof, may be employed (Dexter, T. M. et al. J. Cell. Physiol. 91, 335, 1977; Witlock, C. A. and Witte, O. N. Proc. Natl. Acad. Sci. U.S.A. 79, 3608-3612, 1982).

WO 2006/085482, for example, describes a technique for amplifying a hematopoietic stem cell ex vivo. By using the amplified hematopoietic stem cell or a stem cell of each of various tissues, a transplantation therapy and a gene therapy for a patient with a variety of intractable hematologic diseases or a variety of organ diseases can be conducted.

Various factors can also be tested for use in stimulation of proliferation in vitro, including but not limited to interleukin-3 (IL-3), granulocyte-macrophage (GM)-colony stimulating factor (CSF), IL-1 (hemopoietin-1), IL-4 (B cell growth factor), IL-6, alone or in combination.

In some embodiments, processing concentrates or isolates the stem cells in the sample. In preferred embodiments, after processing, the processed sample contains a sufficient amount of stem cells for the successful transplantation of a patient.

In some embodiments, the stem cells are processed before their storage. In other embodiments, the stem cells are subjected to processing after they are stored. According to this embodiment, the stem cells are processed upon a requirement to use them to treat an individual in need, and they are processed to reach a level sufficient to treat that individual's condition. A sufficient amount of stem cell for transplantation purposes means that enough stem cells are present in the product to successfully treat a person in need of stem cell transplantation.

### Storage

The obtained stem cells can be stored under appropriate conditions to keep them viable and functional. In some embodiments, stem cells units from a certain donor and/or stem cells units of induced pluripotent stem cells are stored in cold conditions.

The freezing of cells is ordinarily destructive. On cooling, water within the cell freezes. Injury then occurs by osmotic effects on the cell membrane, cell dehydration, solute concentration, and ice crystal formation. As ice forms outside the cell, available water is removed from solution and withdrawn from the cell, causing osmotic dehydration and raised solute concentration which eventually destroys the cell. These injurious effects can be circumvented by (a) use of a cryoprotective agent, (b) control of the freezing rate, and (c) storage at a temperature sufficiently low to minimize degradative reactions.

For example, considerations and procedures for the manipulation, cryopreservation, and long-term storage of hematopoietic stem cells, particularly from bone marrow or peripheral blood, are known in the art.

Some methods are reviewed by Gorin, N. C. in Clinics In Haematology 15, 19-48, 1986. Other exemplary methods of cryopreservation of viable cells, or modifications thereof, are available and envisioned for use (e.g., cold metal-mirror techniques; U.S. Pat. No. 4,199,022; U.S. Pat. No. 3,753,357; U.S. Pat. No. 4,559,298). U.S. Pat. No. 6,310,195 discloses a method for preservation of pluripotent progenitor cells, as well as totipotent progenitor cells based on a use of a specific protein. U.S. Pat. No. 5,873,254 discloses device and methods for multigradient directional cooling and warming of biological samples. This method, as well as other methods and devices known in the art for cryopreservation may be used with the cells according to the present invention.

Cryoprotective agents which can be used include but are not limited to dimethyl sulfoxide (DMSO), glycerol, polyvinylpyrrolidine, polyethylene glycol, albumin, dextran, sucrose, ethylene glycol, i-erythritol, D-ribitol, D-mannitol, D-sorbitol, i-inositol, D-lactose, choline chloride, amino acids, methanol, acetamide, glycerol monoacetate, and inorganic salts.

Freezing systems may include but are not limited to a conventional freezer or a chamber holding a freezing medium such as liquid nitrogen, dry ice, frozen water, etc.

In some embodiments, the stored stem cells may be cryogenically preserved but any method of storing stem cell for a long duration of time may be used, e.g., including storage of cells with amino acids, inosine, adenine, etc. Any storage method may be used in this invention providing that the stored product retain viability for the therapeutic purposes discussed in this invention.

According to various embodiments, agents that enhance cell survival during freezing and thawing are added to the cell deposits.

In some preferred embodiments, the stem cells are stored in a cryogenic tank that can be accessed at a later time, as needed. In some embodiments, samples will be stored in cryo-tanks denoting their types, tissue of origin, time of collection and/or donor identity.

In some typical embodiments, the stored stem cells are indexed in a manner for reliable and accurate identification and retrieval upon request. Any conventional indexing system is suitable for the purposes of the present invention, as long as it is reliable and accurate. For example, each container for each donated unit may be marked with alphanumeric codes, bar codes, or any other cognizable method or combinations thereof.

In some embodiments, the information about the stem cells stored in the bank is recorded in an accessible and readable database and/or indexing system. The recorded information may include, but is not limited to, the type of stem cells, their tissue of origin, the date of their collection, the donor identity and any other identifying information that was obtained from characterization assays, for example from characterization assays as described above.

This indexing system can be managed in any way known in the art, for example, manually or non-manually. In some embodiments, a computer and conventional software can be used.

In some embodiments, there is no upper limit on the number of stem cell units that can be stored in the bank.

The storage facility may include means for any method of organizing, and indexing the stored products. In some embodiments, automated robotic systems are used for the retrieval and/or the manipulation of the stored stem cells.

More than one storage facility may be used to store the stem cells. These facilities may each be at a different location.

### Reconstitution of Stored Cells and Use

It is understood that the bank of the present invention provides wide protection for many possible pathological conditions an individual may encounter during the individual's lifetime. The large variety of stem cells types confers a wide option for protection for many pathological conditions that require organ repair. The periodical donations ensure a sufficient amount of stem cells available for use. Furthermore, by implementing personalized medicine approaches, optimized treatments, tailored to a person's characteristics, are envisioned.

Upon request, stem cells can be reconstituted and provided to an individual in need thereof. In the case of cryopreservation, cells can be reconstituted and used clinically by careful thawing, under controlled conditions for thawing.

Frozen cells are preferably thawed quickly (for example, in a water bath maintained at 37-41° C.) and chilled immediately upon thawing. In particular, the vial containing the frozen cells can be immersed up to its neck in a warm water bath; gentle rotation will ensure mixing of the cell suspension as it thaws and increase heat transfer from the warm water to the internal ice mass. As soon as the ice has completely melted, the vial can be immediately placed in ice.

It should be noted that the number of nucleated cells in a stored stem cells sample may change following a freezing/thawing procedure. Consequently, when reviewing stem cell transplantation data, it is instructive to note whether nucleated cell count was measured before or after thawing the sample. For example, in Sanz et al., the median proportion of nucleated cells lost during thawing was thirty percent. See Sanz et al., 2001, “Standardized, unrelated donor cord blood transplantation in adults with hematologic malignancies,” Blood 98, p. 2332.

The stem cells stored in the bank of the present invention can by used for any application that utilizes stem cells, including applications currently known as well as new applications that may be developed in the future.

Non-limiting examples of suitable applications of stem cells in cell therapy include organ and tissue therapy applications using undifferentiated cells, organ and tissue therapy applications using differentiated cell cultures, forming new blood vessels in damaged tissue, cell therapy applications for neuronal disorders, cell therapy applications for bone and/or cartilage injuries, cell therapy applications for liver disorders, cell therapy applications for heart disorders, cell therapy applications to treat diseases or disorders of the pancreas and gene therapy applications.

Diseases that can be treated by the present methods include, but are not limited to, those that can be treated by tissue regeneration or reconstitution, by protein replacement, or by coagulation factors. Such diseases include diseases associated with defective biological processes such as cardiac ischemia, osteoporosis, chronic wounds, diabetes, neural degenerative diseases, neural injuries, bone or cartilage injuries, ablated bone marrow, anemia, liver diseases, hair growth, teeth growth, retinal disease or injuries, ear diseases or injury, muscle degeneration or injury, plastic surgery. In addition, the treatment methods may be applied to cosmetic therapies including, filling of skin wrinkles, supporting organs, supporting surgical procedures, treating burns, and treating wounds, for example.

In some embodiments, stem cells stored in the bank are used for autologous transplantations. In other embodiments, stem cells stored in the bank are used for allogeneic transplantations.

In some embodiments, a specific insurance program regularizes allogeneic transplantations.

In some embodiments, an optimal combination of stem cell types can be selected from the reservoir of cells, in order to treat a certain pathological condition.

In some embodiments, stem cells stored in the bank serve as a basis for personalized medicine. In some specific embodiments, the cells form the basis for a personalized medicine which is based on the ability to cure and regenerate parts of the body that no longer function and/or were damaged by new cells that replace the damaged ones.

### Enrolment and Allocation System

The basis for the stem cell bank of the present invention is the enrolment of donors, periodical collection and storage of stem cells donations therefrom, and allocation of donated stem cells to individuals in need (whether in an autologous or allogeneic manner), and/or for research and pharmaceutical development applications.

In some preferred embodiments, enrollment is performed before a child is born. In other embodiments, a donor is enrolled after birth. In additional embodiments, a donor is enrolled as an adult.

In some embodiments, upon enrolment a record is created for the donor. In other embodiments, a record is created for the donor's family as well. The record may include any relevant information about the donor and/or the donor's family. In some embodiments, the information includes genetic information.

In some embodiments, upon enrollment a donor can elect whether stem cells taken from that donor will be made available for allogeneic uses. Such elections are stored in the subscription information.

Stem cells are collected from individuals, processed and banked.

Donations to be deposited in the bank of the present invention can be collected from individuals at any facility enabling such a procedure. In some embodiments, donations are collected in a hospital. In other embodiments, donations are collected directly at the bank facility. Donations collected outside the storage facility can be delivered and/or transported to the bank after their collection.

Extraction of stem cells from the tissues obtained from the individual can be performed at the collection facility or at the bank.

Characterization of the obtained stem cells can be performed at the collection facility or at the bank. All relevant information about a stem cells unit can be stored in a database. In some embodiments, a computer-based database and/or allocation system are provided.

Stem cells are typically sorted and stored according to various categories. In some embodiments, the stem cells are sorted according to their type, their tissue of origin, date of their collection and donor identity.

In some embodiments, the stem cells are arranged in stem cell units. In some embodiments, a stem cell unit is defined by the number of nucleated cells in a unit. Non-limiting examples of other metrics for defining stem cells unit include the number of cells collected or thawed which expresses a certain marker and the number of colony forming cells collected or thawed.

In some embodiments, periodical donations to the bank are initiated by the donor. In other embodiments, periodical donations to the bank are initiated by the bank.

In some embodiments, a fee is charged for the isolation, storage and/or dispensing of the cells.

In some embodiments, after a stem cell sample or stem cell unit is recorded into an indexing system, it will be available for matching purposes.

In some embodiments, the information stored with each sample is searchable and identifies the sample in such a way that it can be efficiently located and supplied to an individual in need thereof.

The number of units/samples available for allocation to an individual depends on the amount of donations made by that individual.

In the case of an allogeneic transplantation donation, a matching test may be performed between the donor and recipient in need. For the purposes of this invention, matching indicates that the stem cells are suitable for transplantation into a specific individual. A recipient in need and/or tissues of a recipient in need is characterized for specific characteristics that are important in order to determine a match between this recipient and a certain stem cells unit or sample. For example, the presence of certain cell markers which are typical to each tissue. After characterization, matching stem cells from the stem cells available for allogeneic transplantations (for example, stem cells that were authorized by their donor for use by others) is retrieved and can be used to treat the recipient in need thereof.

In some embodiments, the search can use an appropriate matching algorithm.

Any matching criteria and/or matching assays, currently known or new ones that will be developed in the future are under the scope of activities performed at the bank of the present invention.

The transplantation process will be performed in any facility enabling such a procedure, for example, a hospital.

### Stem Cells Compositions

According to an aspect of the present invention, compositions of stem cells are provided, the compositions comprising mixtures of different types of stem cells for use in stem cell therapy, wherein the stem cells originate from a single individual. In some embodiments, the stem cells were obtained from a plurality of donations collected periodically over the course of life of the single individual.

### Possible Combinations

In some embodiments, the composition comprises mixtures of adult stem cells of more than one type. In some embodiments, the composition of stem cells comprises embryonic and adult stem cells. In some embodiments, the composition comprises adult stem cells of various types, mixed in different ratios. In additional embodiments, the composition of stem cells comprises adult stem cells of various types, wherein each type is present in a different dose.

In various specific embodiments, the adult stem cells in the composition can be of various types. Any type of adult stem cells known in the art can be used in the compositions of the present inventions, some are specified above.

A non-limiting example of adult stem cells mixtures includes mixtures of mesenchymal and fat-derived stem cells.

In some embodiments, the composition of stem cells comprises induced pluripotent stem cells. Any type of IPSC can be used in the compositions of the present invention.

In additional embodiments, the composition of stem cells comprises IPSCs of various types, mixed in different ratios. In yet additional embodiments, the composition of stem cells comprises IPSCs of various types wherein each type is present in a different dose.

In some embodiments, the differentiation potential of the stem cells present in the compositions of the present invention can be selected from pluripotent, multipotent, oligopotent and unipotent.

In some specific embodiments, the types of stem cells to be mixed and/or the ratio between them are determined and optimized according to the pathological condition of an individual in need. By optimizing the ratio between the different types of stem cell in a composition, an improved therapeutic effectiveness may be achieved.

In some embodiments, adult stem cells in the compositions are of an autologous origin. In other embodiments, they are of an allogeneic origin.

In some embodiments, IPSCs in the compositions are of an autologous origin. In other embodiments, they are of an allogeneic origin.

In some embodiments, the stem cells present in the composition are obtained from periodical donations collected throughout the lifetime of the individual in need thereof.

In some embodiments, the composition of stem cells comprises mixtures of adult stem cells of various types, provided in different dosages forms.

The stem cells compositions of the present invention can be administered to a patient in any known route of administration suitable for stem cells applications. These include local as well as systemic administration. In preferred embodiments, the compositions are administered to patients by a method that allows the stem cells to reach the sites needed for the composition to generate the desired therapeutic effect. Non-limiting examples include intravenous, injection directly into specific organs and injection directly to the site of action.

In a preferred embodiment, compositions of the present invention are administered to a patient in therapeutically effective amount.

### Additional Ingredients

The compositions of the present invention can further comprise a conductive, support material. The term “conductive material” as used herein refers to a material which helps convey the stem cells to the site of tissue defect. Use of a conductive material aims to enhance the therapeutic effect of the stem cells, by providing a milieu conducive to their survival or by aiding in retention of the cells at the site in need of repair. Non-limiting examples of such conductive materials include paste (e.g. amorphous calcium phosphate paste, hydroxy apatite, calcium sulfate paste and demineralized bone), a natural or synthetic suitable scaffold (e.g. a fibrin matrix), a viscous milieu based on a biopolymer such as hyaluronic acid or a combination of these materials. Examples for suitable fibrin matrices may be found for example in U.S. Pat. No. 7,009,039, WO 2004/067704 and WO 2006/008748.

The composition of the present invention may also include an inductive material that would enhance expansion of the stem cells present in the composition in-vivo. The term “inductive material” as used herein refers to a substance which enhances the therapeutic (regenerative) effect of the stem cells. The inductive material may act directly to promote tissue regeneration or it may act by promoting proliferation of the stem cells or both. Non-limiting examples of inductive agents include growth factors. Examples for growth factors include: vascular endothelial growth factor (VEGF), fibroblast growth factor (FGF), epidermal growth factor (EGF), insulin-like growth factor 1 (IGF1), bone morphogenetic proteins (BMP), and transforming growth factor (TGF). In one embodiment the stem cells are expanded ex-vivo with FGF, preferably with FGF2. Growth factors may be administered at a wide range of concentrations, depending on the type of bone defect, the age of the patient, body weight, the route of administration, etc.

The composition of the present invention may further include a pharmaceutically acceptable carrier. Non-limiting examples of carriers, known in the art, which may be used according to the present invention include:
Extracellular matrix components such as collagens, glycoprotein, proteoglycans, glycans as hyaluronic acid fibrin, and others and a combination of these. These types of carriers may take the form of gels or solid structures that may be porous of sizes ranging from nanometers to centimeters.
Natural or synthetic apatites such as hydroxyapatite, deproteinized bone particles, frozen dried bone particles, demineralized frozen died bone particle, calcium phosphates as tricalcium phosphate, calcium sulfates, calcium carbonates and other natural and synthetic salts known in the art and combinations of these.
Synthetic organic polymers as polylactates, polyfumarates, polyglycolics and others known in the art or their combination.

It is within the scope of the present invention to utilize the compositions of the present invention together with any matrices or scaffolds as are known in the art.

### Potential Uses

Essentially all of the uses known or envisioned in the prior art for stem cells can be accomplished with the stem cells and compositions of stem cells of the present invention. These uses include diagnostic, prophylactic and therapeutic techniques.

In addition, the stored stem cells may be used to establish stem cell lines for use in drug discovery, drug testing and drug development.

Non-limiting examples of tissues and organs that can be treated and/or repaired or regenerated: bone, cartilage (hyaline and articular), blood vessels, ligaments, tendons, myocardium, hematopoietic system, muscular tissue, dermis, heart valves, the mesenchymal part of intestinal tubes (e.g. column, esophagus, ileum, rectum), urogenital vessels (e.g. urethra, ureter, urinary bladder), periodontal tissues (e.g. alveolar bone, periodontal ligament, cementum, gingiva), dentin and any other mesenchymal tissue or mesenchymal component of any tissue of the adult or fetal organism.

Differentiated cells of the present invention can be used for tissue reconstitution or regeneration in a human patient in need thereof. The cells are administered in a manner that permits them to graft to the intended tissue site and reconstitute or regenerate the functionally deficient area.

Differentiated cells of present invention can also be used for transplant therapy. For example, neural stem cells can be transplanted directly into parenchymal or intrathecal sites of the central nervous system, according to the disease being treated (U.S. Pat. No. 5,968,829). The efficacy of neural cell transplants can be assessed in a rat model for acutely injured spinal cord as described by McDonald et al. (Nat. Med. 5, 1410, 1999).

Non-limiting examples of diseases that can be treated by the present compositions include all those diseases already listed above, in addition to the following:

Diseases treatable by stem cell transplantation stem cell disorders such as (e.g., aplastic anemia, Fanconi anemia, paroxysmal nocturnal hemoglobinuria), acute leukemias (e.g., acute lymphoblastic leukemia, acute myelogenous leukemia, acute biphenotypic leukemia, acute undifferentiated leukemia), chronic leukemias (e.g., chronic myelogenous leukemia, chronic lymphocytic leukemia, juvenile chronic myelogenous leukemia, juvenile myelomonocytic leukemia), myeloproliferative disorders (e.g., acute myelofibrosis, agnogenic myeloid metaplasia, polycythemia vera, essential thrombocythemia), myelodysplastic syndromes (e.g., refractory anemia, refractory anemia with ringed sideroblasts, refractory anemia with excess blasts, refractory anemia with excess blasts in transformation, chronic myelomonocytic Leukemia), lymphoproliferative disorders (e.g., non-Hodgkin's lymphoma, Hodgkin's disease, prolymphocytic Leukemia) inherited erythrocyte abnormalities (e.g., beta thalassemia major, pure red cell aplasia, sickle cell disease), liposomal storage diseases (e.g., mucopolysaccharidoses, Hurler syndrome, Scheie syndrome, Hunter's syndrome, Sanfilippo syndrome, Morquio syndrome, Maroteaux-Lamy syndrome, Sly syndrome, beta-glucuronidase deficiency, adrenoleukodystrophy, mucolipidosis II, Krabbe disease, Gaucher's disease, Niemann-Pick disease, Wolman disease, metachromatic leukodystrophy), histiocytic disorders (e.g., familial erythrophagocytic lymphohistiocytosis, histiocytosis-X, hemophagocytosis), phagocyte disorders (Chediak-Higashi syndrome, chronic granulomatous disease, neutrophil actin deficiency, reticular dysgenesis) congenital immune system disorders (e.g., ataxia-telangiectasia, kostmann syndrome, leukocyte adhesion deficiency, DiGeorge syndrome, bare lymphocyte syndrome, Omenn's syndrome, severe combined immunodeficiency (SCID), SCID with adenosine deaminase deficiency, absence of T and B Cell SCID, absence of T Cell, normal B Cell SCID, common Variable Immunodeficiency, Wiskott-Aldrich syndrome, x-linked lymphoproliferative disorder, inherited platelet abnormalities (e.g., amegakaryocytosis/congenital thrombocytopenia), plasma cell disorders, (e.g., multiple myeloma, plasma cell leukemia, Waldenstrom's macroglobulinemia), other inherited disorders (e.g., Lesch-Nyhan syndrome, cartilage-hair hypoplasia, Glanzmann Thrombasthenia, Osteopetrosis), and other malignancies (e.g., breast cancer, Ewing sarcoma, neuroblastoma, and renal cell carcinoma).

Stem cells are also expected to have an anti-inflammatory effect when administered to an individual experiencing inflammation. In a preferred embodiment, stem cells may be used to treat any disease, condition or disorder resulting from, or associated with, inflammation. The inflammation may be present in any organ or tissue, for example, muscle; nervous system, comprising the brain, spinal cord and peripheral nervous system; vascular tissues, comprising cardiac tissue; pancreas; intestine or other organs of the digestive tract; lung; kidney; liver; reproductive organs; endothelial tissue, or endodermal tissue.

Stem cells may also be used to treat immune-related disorders, particularly autoimmune disorders, comprising those associated with inflammation. Examples include diabetes, amylotrophic lateral sclerosis, myasthenia gravis, diabetic neuropathy or lupus. Cord blood or cord blood-derived stem cells may also be used to treat acute or chronic allergies, e.g., seasonal allergies, food allergies, allergies to self-antigens, etc.

In certain embodiments, the disease or disorder includes, but is not limited to, any of the diseases or disorders disclosed herein, comprising, but not limited to aplastic anemia, myelodysplasia, myocardial infarction, seizure disorder, multiple sclerosis, stroke, hypotension, cardiac arrest, ischemia, inflammation, age-related loss of cognitive function, radiation damage, cerebral palsy, neurodegenerative disease, Alzheimer's disease, Parkinson's disease, Leigh disease, AIDS dementia, memory loss, amyotrophic lateral sclerosis (ALS), ischemic renal disease, brain or spinal cord trauma, heart-lung bypass, glaucoma, retinal ischemia, retinal trauma, lysosomal storage diseases, such as Tay-Sachs, Niemann-Pick, Fabry's, Gaucher's, Hunter's, and Hurler's syndromes, as well as other gangliosidoses, mucopolysaccharidoses, glycogenoses, inborn errors of metabolism, adrenoleukodystrophy, cystic fibrosis, glycogen storage disease, hypothyroidism, sickle cell anemia, Pearson syndrome, Pompe's disease, phenylketonuria (PKU), porphyrias, maple syrup urine disease, homocystinuria, mucoplysaccharidosis, chronic granulomatous disease and tyrosinemia, Tay-Sachs disease, cancer, tumors or other pathological or neoplastic conditions.

In other embodiments, the stem cells may be used in the treatment of any kind of injury due to trauma, particularly trauma involving inflammation. Examples of such trauma-related conditions include central nervous system (CNS) injuries, comprising injuries to the brain, spinal cord, or tissue surrounding the CNS injuries to the peripheral nervous system (PNS); or injuries to any other part of the body. Such trauma may be caused by accident, or may be a normal or abnormal outcome of a medical procedure such as surgery or angioplasty. Trauma may also be the result of the rupture, failure or occlusion of a blood vessel, such as in a stroke or phlebitis. In specific embodiments, the stem cells may be used in autologous or heterologous tissue regeneration or replacement therapies or protocols, comprising, but not limited to treatment of corneal epithelial defects, cartilage repair, facial dermabrasion, mucosal membranes, tympanic membranes, intestinal linings, neurological structures (e.g., retina, auditory neurons in basilar membrane, olfactory neurons in olfactory epithelium), burn and wound repair for traumatic injuries of the skin, or for reconstruction of other damaged or diseased organs or tissues.

Additional examples include bone fractures in general and vertebral stabilization in particular, osteoporosis, ligament rupture, osteoarthritis, any arthritis of autoimmune origin, traumatic articular cartilage damage, myocardial infarction, heart failure, mitral or aortic valves insufficiency, coronary insufficiency, diabetes, hepatic insufficiency or failure, reflux, fecal incontinence, urinary incontinence, renal insufficiency or failure, emphysema, Parkinson disease, muscular atrophies, muscular dystrophies, amyotrophic lateral sclerosis, multiple sclerosis and other demyelinating diseases, myasthenia gravis, polymyositis, loss of brain tissue caused by cerebrovascular diseases or encephalitis or meningitis, insufficiency and failure of endocrine glands (e.g. hypothyroidism, hypoparathyroidism, pituitary and adrenal hypofunction), acquired or induced failure of the hematopoietic system, periodontal disease and loss of any other tissue(s) mass and function caused by degenerative, inflammatory, proliferative, infectious, malignant diseases, trauma and aging.

In some embodiments, the compositions comprising stem cells mixtures can be used for the treatment of injured joints, chronic ulcers, large burns, corneal damage, neural damage, neuro-degenerative diseases and/or cancer.

In some embodiments, the compositions comprising stem cells mixtures can be used for the regeneration of a tissue that can be selected from cartilage, heart muscle, liver, insulin secreting Langerhans cells and/or nerve cells.

It is understood that the compositions of the present may be used alone or in conjunction with other compositions and methods for tissue regeneration as are known in the art.

The compositions of the present invention can be used in an autologous and/or allogeneic manner.

### Personalized Medicine

Personalized medicine involves the systematic use of information about each individual patient to select or optimize the patient's preventative and therapeutic care. In the modern conception of personalized medicine, the tools provided to the physician are more precise, probing not just the obvious, such as a tumor on a mammogram or cells under a microscope, but the very molecular makeup of each patient.

The ambition of personalized medicine is to be able to provide a patient with a more precise therapy, a therapy that matches the individual's properties down to the molecular properties. The use of autologous stem cells as the basis for curing and regenerating parts of the body, provides a personalized medicine approach to regenerative medicine. Under certain circumstances allogeneic donations may be acceptable for use. Furthermore, personalized medicine implications provide the option for optimized treatments, tailored to the person's characteristics.

According to another aspect, the present invention provides a method of personalized medicine, the method comprising retrieving stem cells stored in a stem cell bank of the present invention, wherein said stem cells originate from a single individual; and administering said stem cells to said individual.

In some embodiments, the method comprises retrieving one or more stem cells units stored in a stem cell bank of the present invention, wherein said one or more stem cell units originate from a single individual, and administering said one or more stem cell units to said individual.

In some embodiments, the stem cells undergo further processing before they are administered to the individual. For example, the cells may be subjected to differentiation procedures.

The provided stem cells, or stem cell units, may be the same or different. In some embodiments, the provided stem cells, or stem cell units, are of the same type. In other embodiments, a combination of stem cells, or stem cell units, of different types is used.

In some embodiments, a composition for use in personalized medicine is provided, the composition comprising stem cells reconstituted from a bank of the present invention.

In some embodiments, after diagnosis of a certain condition that requires the application of stem cells therapy, an analysis is performed to determine the best combination of stem cells to be included in the composition that will be administered to a patient.

1. A stem cell bank comprising stem cells from a plurality of individuals, wherein the stem cells originate from a plurality of donations collected periodically from each individual over the course of the individual's life.

2. The stem cell bank of claim 1, wherein a plurality of donations are collected periodically from an individual over the course of the individual's life, sorted and stored for future use.

3. The stem cell bank of claim 1, wherein the plurality of donations comprises a first donation at birth and subsequent donations as the individual grows and matures.

4. The stem cell bank of claim 1, wherein donations are collected from individuals between the ages 20 to 50.

5. The stem cell bank of claim 1, wherein the plurality of donations collected from said each individual are obtained from different sources.

6. The stem cell bank of claim 5, wherein the source of stem cells comprises at least one source selected from the group consisting of umbilical cord blood, cord matrix, placental blood, bone marrow, fat, peripheral blood, blood buffy coat, amniotic fluid, skin, kidney, liver, muscle, neural tissue, tooth pulp, mucosa, foreskin, cardiac tissue, bone, cartilage, hair roots and mammary glands.

7. The stem cell bank of claim 1, wherein the plurality of donations collected from said each individual comprises stem cells of different types.

8. The stem cell bank of claim 1, wherein the stored stem cells comprise at least one type selected from the group consisting of hematopoietic cells, lineage-committed hematopoietic cells, mesenchymal stem cells, stromal cells, fibroblasts, endothelial progenitor cells, neural stem cells, adipose-derived stem cells, stem cells derived from mucosa, placenta-derived stem cells, amniotic stem cells, cord blood derived stem cells, cord matrix derived stem cells, stem cells derived from foreskin, cardiac stem cells and mammary stem cells.

9. The stem cell bank of claim 1, wherein the plurality of donations collected from said each individual comprises differentiated somatic cells.

10. The stem cell bank of claim 1, wherein stored cells comprise induced pluripotent stem cells.

11. The stem cell bank of claim 1, wherein the differentiation potential of the stem cells stored in the bank is selected from the group consisting of pluripotent, multipotent, oligopotent and unipotent.

12. The stem cell bank of claim 1, wherein information about each donation is recorded.

13. The stem cell bank of claim 12, wherein the recorded information comprises at least some data selected from the group consisting of the type of cells, their tissue of origin, the date of their collection, the identity of the donor and results obtained from characterization assays.

14. The stem cell bank of claim 13, wherein the characterization assays comprise at least one assay selected from the group consisting of HLA typing, determining the presence of specific markers, determining specific SNP alleles and performing a nucleated cell count.

15. The stem cell bank of claim 2, wherein collected cells are sorted according to at least one criterion.

16. The stem cell bank of claim 15, wherein the criteria for sorting the cells comprise at least one parameter selected from their type, their tissue of origin, the date of their collection and the donor identity.

17. The stem cell bank of claim 1, wherein the stem cells are stored under appropriate conditions to keep the stem cells viable and functional.

18. The stem cell bank of claim 17, wherein the stem cells are stored under cryopreservation conditions.

19. The stem cell bank of claim 1, wherein stem cells stored in the bank are for autologous use.

20. The stem cell bank of claim 19, wherein the amount of stem cells available for allocation to an individual depends on the amount of donations made by that individual.

21. The stem cell bank of claim 1, wherein stem cells stored in the bank are for allogeneic use.

22. The stem cell bank of claim 21, wherein the stem cells stored in the bank are used for drug discovery and development.

23. The stem cell bank of claim 1, wherein the stem cells stored in the bank are arranged in stem cell units.

24. The stem cell bank of claim 1, wherein stem cells are subjected to further processing after their collection.

25. The stem cell bank of claim 24, wherein the collected stem cells are processed in order to achieve therapeutic levels.

26. A method of stem cell banking, the method comprising periodically collecting a plurality of donations from an individual over the course of the individual's life.

27. The method of claim 26, wherein collecting a plurality of donations from an individual comprises collecting stem cells from more than one source.

28. The method of claim 26, wherein collecting a plurality of donations from an individual comprises collecting stem cells of more than one type.

29. The method of claim 26, wherein collecting a plurality of donations from an individual comprises collecting differentiated somatic cells.

30. A method of personalized medicine, the method comprising providing stem cells stored in the stem cell bank of claim 1, wherein said stem cells originate from a single individual; and administering said stem cells to said individual.

31. A composition comprising mixtures of different types of stem cells for use in stem cell therapy, wherein the stem cells originate from a single individual.

32. The composition of claim 31, wherein the stem cells were obtained from a plurality of donations collected periodically over the course of life of said single individual.

33. The composition of claim 31, wherein the composition further comprises embryonic stem cells.

34. The composition of claim 31, wherein the composition comprises adult stem cells of more than one type.

35. The composition of claim 34, wherein the composition comprises adult stem cells of more than one type mixed in different ratios.

36. The composition of claim 34, wherein the type of adult stem cells in the composition comprises at least one type selected from the group consisting of hematopoietic cells, lineage-committed hematopoietic cells, mesenchymal stem cells, stromal cells, fibroblasts, endothelial progenitor cells, neural stem cells, adipose-derived stem cells, stem cells derived from mucosa, placenta-derived stem cells, amniotic stem cells, cord blood derived stem cells, cord matrix derived stem cells, stem cells derived from foreskin, cardiac stem cells and mammary stem cells.

37. The composition of claim 31, wherein the composition comprises induced pluripotent stem cells.

38. The composition of claim 37, wherein the composition comprises induced pluripotent stem cells of more than one type, mixed in different ratios.

39. The composition of claim 31, wherein the differentiation potential of the stem cells present in the composition is selected from the group consisting of pluripotent, multipotent, oligopotent and unipotent.

40. The composition of claim 31, wherein the stem cells are of an autologous origin.

41. The composition of claim 31, wherein the stem cells are of an allogeneic origin.

42. Use of a composition comprising mixtures of different types of stem cells in stem cell therapy, wherein the stem cells originate from a single individual.

43. Use of a composition comprising mixtures of different types of stem cells in personalized medicine, wherein the stem cells originate from a single individual.
